(19) United States
(12) Patent Application Publication
(10) Pub. No.: US 2007/0141613 A1
(43) Pub. Date: Jun. 21, 2007

(54) Parametric imaging of gene expression in biopsy samples utilizing the reverse transcriptase-polymerase chain reaction method

(75) Inventor: Farhad Daghighian, Santa Monica, CA (US)

Correspondence Address: KOPPEL, PATRICK & HEYBL, 555 ST. CHARLES DRIVE, SUITE 107, THOUSAND OAKS, CA, 91360, US

(73) Assignee: INTRAMEDICAL IMAGING, LLC

(21) Appl. No.: 11/639,487

(22) Filed: Dec. 15, 2006

Related U.S. Application Data
(60) Provisional application No. 60/752,348, filed on Dec. 20, 2005.

(51) Int. Cl. C12Q 1/68; G06F 19/00; G06T 7/00

(52) U.S. Cl. 435/6; 702/20; 382/128

(57) ABSTRACT

A method is described for generating and showing the profile of one or more genes across a tissue sample. Tissue removed from the body is sliced in to thin sheets and those sheets are then divided into small portions each portion being identified as to a location in the sheet. The image of the thin slice and the position of each small portion thereof is recorded in a computer in a manner that can generate an image of the slice. Each small portion is subject to RT-PCR to identify the presence and quantity of one or more genes therein. The portion-specific data is then entered into the computer and an image of the slice is generated showing the gene specific characteristics of each small portion. The result is a parametric image of the entire slice which allows the visualization of the gene expression within each portion which can then be compared with other images of the same or adjacent tissue.

This application claims the benefit of U.S. Provisional Application No. 60/752,348, filed Dec. 20, 2005.

Described is a technique for characterizing the disease state of excised tissue, such as a tumor removed by a biopsy procedure, which uses RT-PCR techniques to identify one or more target genes in multiple discrete portions of the tumor, resulting in a graphical or digital representation of the target genes across the tissue sample being studied.

### BACKGROUND

PCR (Polymerase Chain Reaction) is a technique used in molecular genetics that permits the analysis of any short sequence of DNA (or RNA) without having to clone it. PCR is used to reproduce (amplify) selected sections of DNA. Previously, the amplification procedure which utilized bacteria took weeks. Using PCR the procedure is performed in test tubes and takes only a few hours. PCR is highly efficient so that a large numbers of copies of the DNA can be made. In addition, PCR uses the same molecules that nature uses for copying DNA:
Two “primers” that flag the beginning and end of the DNA stretch to be copied;
An enzyme called polymerase that walks along the segment of DNA, reading its code and assembling a copy; and
A pile of DNA building blocks that the polymerase needs to make that copy.

Three major steps are involved in the PCR procedure and these three steps are repeated numerous times, for example 30 or 40 cycles. The cycles are performed using an automated cycler, which rapidly heats and cools the test tubes containing the reaction mixture so that each of the steps can be performed in the intended order. Each of the steps, namely denaturation (alteration of structure), annealing (joining), and extension, takes place at a different temperature:
1. In Denaturation, performed at 94° C., the double DNA strand is separated and opened into single-stranded DNA.
2. This is followed by Annealing, performed at 54° C., in which hydrogen bonds are form and broken between the single-stranded “primer” and the single-stranded “template”, the template providing the pattern to be copied. The more stable bonds last longer and on the length of double-stranded DNA, which comprises the joined primer and template, the polymerase attaches and starts copying the template.
3. Extension is then performed at 72° C., which is the optimum temperature for the polymerase to work best. During the extension phase the attraction created by the hydrogen bonds of the primers to the template is stronger than the forces breaking these attractions. The result is that bases complementary to the template become coupled to the primer.

Because PCR does not require that the original DNA be copied to be pure or abundant, PCR has found widespread and innumerable uses. These uses include but are not limited to diagnosing genetic diseases, conducting DNA fingerprinting, finding bacteria and viruses, studying human evolution, cloning the DNA of an Egyptian mummy, etc. As a result PCR has become an essential tool for biologists, DNA forensics labs and many other laboratories that work with genetic material.

Kary Mullis, the inventor of PCR, has written in the Scientific American: “Beginning with a single molecule of the genetic material DNA, the PCR can generate 100 billion similar molecules in an afternoon. The reaction is easy to execute. It requires no more than a test tube, a few simple reagents, and a source of heat.”

RT-PCR (Reverse transcriptase-polymerase chain reaction) is a highly sensitive technique for the detection and quantification of mRNA (messenger RNA). The technique consists of two parts:

1) The synthesis of cDNA (complementary DNA) from RNA by reverse transcription (RT), and

2) The amplification of a specific cDNA by the polymerase chain reaction (PCR). RT-PCR has been approved by the FDA as a test to measure viral load with HIV. It may also be used with other RNA viruses such as measles, mumps, and metapneumovirus.

Scientists have developed RT-PCR tests for use on biopsy samples to detect tumor gene expression (RNA) and genetic markers (DNA) that are signatures of specific tumor cells in cancer patients. It is also possible to directly analyze the DNA obtained from a small amount of paraffin-embedded tumor tissue sections (20 microns) by Laser Capture Microdissection (LCM). The LCM allows the assessment of less than about 50 tumor cells cut out from a tumor lesion and determine their molecular profile. (Takeuchi H, Morton D L, Kuo C, Turner R R, Elashoff D, Elashoff R, Taback B, Fujimoto A, Hoon D S B. “Prognostic Significance Of Molecular Upstaging Of Paraffin-Embedded Sentinel Lymph Nodes In Melanoma Patients, J. Clin. Oncol., 22, pp2671-2680 (2004). The normal technique is to profile a biopsy sample by subjecting the whole sample to analysis to identify multiple different genes in the sample.

### SUMMARY OF THE INVENTION

The invention is directed to showing the profile of one or more genes across the tissue sample. Tissue removed from the body is sliced in to thin sheets and those sheets are then divided into small portions each portion being identified as to a location in the sheet. The image of the thin slice and the position of each small portion thereof is recorded in a computer in a manner that can generate an image of the slice. Each small portion is subject to RT-PCR to identify the presence and quantity of one or more genes therein. The portion-specific data is then entered into the computer and an image of the slice is generated showing the gene specific characteristics of each small portion. The result is a parametric image of the entire slice which allows the visualization of the gene expression within each portion which can then be compared with other images of the same or adjacent tissue.

### DESCRIPTION OF THE FIGURES

FIG. 1 is a schematic representation of a thin slice of a tumor specimen cut into small cubes.

FIG. 2 is a parametric image of the tumor slice of FIG. 1 showing, in gray scale, different levels of expression of a specific gene in the various portions of the tumor.

### DETAILED DESCRIPTION OF THE INVENTION

A purpose of the invention is to provide a parametric image of a tissue sample, with the gray scale of the image representing the level of one or more particular genes expressed as measured by RT-PCR, for comparison with other types of images of the same or adjacent slices of the tissue sample. Superimposing or comparing these parametric images on or with the stained microscopic images of the same or adjacent slice will reveal important biological information. In addition, if the living subject was injected with a radio-labeled tracer, the auto-radiography of slices can be superimposed, revealing correlations between gene expression and uptake of various radio-tracers. Thus, the information obtained from the parametic images of gene expression can be of utility in biological research, drug discovery, and study of tissue samples in biopsy of cancer patients, neurological disorders, or other diseases. Also, in this manner the stage of the tumor can be readily ascertained and active margins can be determined.

Another application of this invention is in bio-medical research involving laboratory animals. For example the uptake of F-18 deoxy-glucose can be mapped using autoradiography in a tissue slice, and then using the parametic image of gene expression, one can study the correlation of different genes (such as glucose transporter gene GLUT-1) with the uptake of flouro-deoxy-glucose.

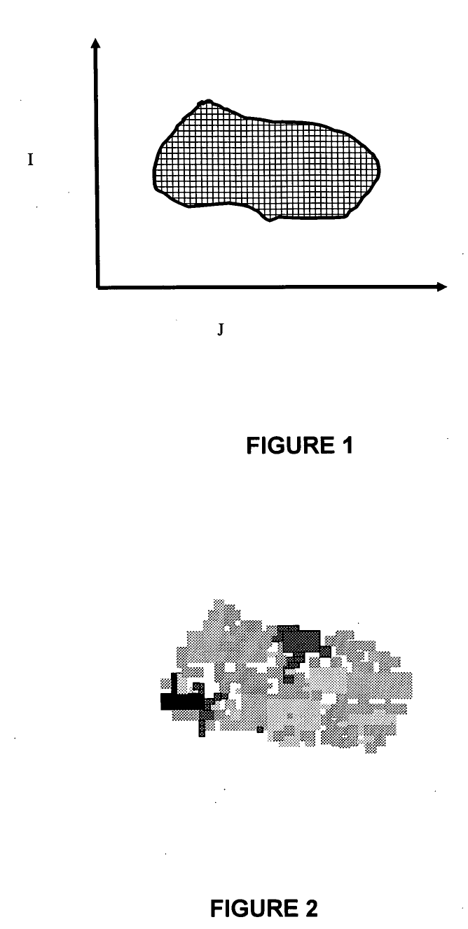

As an example of the procedure:
1—The tissue sample is cut into thin slices, for example 0.01 mm thick slices, and the thin slice is then cut into multiple squares, for example 1×1 mm on a side, such as shown in FIG. 1.
2—A digital picture of the slice is taken to delineate the contour of the tissue sample and the digital image is entered into a computer along with a discrete identification for each square within the sample, for example on the I-J coordinates.
3—The sample is disassembled and RT-PCR analysis is conducted on each square of tissue (0.01×1×1 mm) to identify the level of expression of one or more particular target genes within that cube.
4—The values found for the target gene in each of the squares is then entered into the computer and a parametric image is produced showing the gene-expression values, each in its relative place inside the contour of the tissue sample (some times referred to in the art as Cᵢⱼ).
5—The parametric image formed in this fashion can be superimposed on the visual image as well as other images of the tissue or adjacent tissue before or after excision, such as autoradiography of particular tracers, fluorescent or luminescent images.

FIG. 2 is a parametric image of gene expression generated by the computer for a particular tissue slice. The differences in gray tone indicate various levels of gene expression in the tissue specimen. One skilled in the art will recognize that the dimensions of the small sections set forth above are merely representative and the scope of the invention is limited solely by the claims set forth below.

I claim:

1. A method of characterizing the biological status of a piece of excised tissue comprising:
a) obtaining a sample of tissue, cutting the tissue into thin slices and dividing the thin slice into discrete, identified smaller portions,
b) generating a digital image of the slice of tissue and entering the digital image into a computer along with a discrete identification for each small portion within the sample,
c) disassembling the slice of tissue and subjecting each discrete, identified smaller portions to RT-PCR to identify the level of expression of one or more particular target genes within that small portion, and
d) entering the values obtained for the target gene in each of the small portions into the computer and generating a parametric image showing the gene-expression values, each in its relative place inside the contour of the tissue sample.

2. The method of claim 1 wherein the discrete, identified smaller portions are cubes, the length of each side thereof being about 1 mm.

3. The method of claim 1 wherein one or more additional images of the excised tissue obtained by using different instrumental techniques are also stored in the computer and the parametric image is superimposed on the digital image or the additional image or composite of the digital image and the one or more additional images of the tissue samples.

4. The method of claim 4 wherein the one or more additional images show particular fluorescent, luminescent or radioactive tags within the tissue.
